(19) United States
(12) Patent Application Publication
(10) Pub. No.: US 2008/0076699 A1
(43) Pub. Date: Mar. 27, 2008

(54) Deconjugated alkadienoic acid esters as fragrances and or aroma substances

(75) Inventors: Jakob LEY, Holzminden (DE); Thomas RIESS, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/858,586

(22) Filed: Sep. 20, 2007

(30) Foreign Application Priority Data
Sep. 22, 2006 (DE) 10 2006 044 849.9

(51) Int. Cl. A61K 8/33; A61Q 13/00

(52) U.S. Cl. 512/26

(57) ABSTRACT

The use is described of (i) a fatty acid ester having formula (I) or
(ii) a mixture of two or more different fatty acid esters having formula (I) 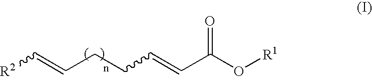 wherein
R¹ is an alkyl radical having 1 to 10 C atoms, a cycloalkyl radical having 3 to 8 C atoms, an alkenyl radical having 2 to 10 C atoms or a cycloalkenyl radical having 3 to 8 C atoms,
R² is an alkyl radical having 1 to 10 C atoms, n denotes 1, 2, 3, 4 or 5, and the two double bonds in the fatty acid radical are mutually independently (E)- or (Z)-configured, as a fragrance and/or aroma substance.

### FIELD OF THE INVENTION

The invention concerns the use of certain deconjugated fatty acid esters which are diunsaturated in the fatty acid radical and are of medium chain length (alkadienoic acid esters) as fragrances and/or aroma substances, and preparations, in particular fragrance and aroma compositions, cosmetic, body care, household, food, consumable or oral hygiene preparations and dermatological or oral pharmaceutical preparations containing said alkadienoic acid esters.

### BACKGROUND OF THE INVENTION

So-called pear esters (2E,4Z)-decadienoic acid ethyl ester and its geometric isomers have long been known as fragrances and aroma substances (cf. U.S. Pat. No. 5,753,473; Chimia 2001, 55, 397-400). Although the scent and flavour impression is characteristic (fruity, Williams pear), overall it is relatively weak. In addition, owing to their completely conjugated double bond system, the compounds are very sensitive to the influence of light and acid (e.g. isomerisation of the double bonds) and can oxidise very easily, giving rise to both degradation products and polymerisation products, which can lower the (sensorial) value of the preparations.

The alkadienals, e.g. (2E,7Z)-nonadienal, that are popularly used for preparations having a fruity, particularly a watery-fruity, scent and/or flavour, are often not sufficiently stable from an applicational perspective. Furthermore, with unsaturated aldehydes there is a risk of their having an irritating and/or sensitising effect on the skin, which severely limits or even prevents their ability to be used.

There is therefore still a need for compounds having a watery-fruity scent and/or flavour which are more stable than the cited aldehydes or completely conjugated alkadienoic acid esters and which in addition can be readily produced in high yields.

### SUMMARY OF THE INVENTION

The stated object is achieved according to the invention by the use of
(i) a fatty acid ester having formula (I) or
(ii) a mixture of two or more different fatty acid esters having formula (I) 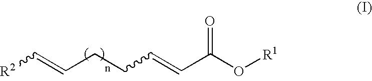
   wherein
R¹ is an alkyl radical having 1 to 10 C atoms,
   a cycloalkyl radical having 3 to 8 C atoms,
   an alkenyl radical having 2 to 10 C atoms or
   a cycloalkenyl radical having 3 to 8 C atoms,
R² is an alkyl radical having 1 to 10 C atoms,
n denotes 1, 2, 3, 4 or 5,
   and
   the two double bonds in the fatty acid radical are mutually independently (E)- or (Z)-configured,
   as a fragrance and/or aroma substance.

Preferred according to the invention is a use of the type specified above, wherein the fatty acid ester having formula (I) or at least one or each fatty acid ester having formula (I) in the mixture comprises
(i) at most 18 C atoms and at least 9 C atoms,
(ii) at most 16 C atoms and at least 9 C atoms
   and/or
(iii) at most 16 C atoms and at least 10 C atoms.

Alkyl radicals within the meaning of the invention are linear or branched, saturated hydrocarbon radicals having 1 to 10, preferably 1 to 4 C atoms, preferably methyl, ethyl, 1-propyl, 2-propyl, 1-butyl, 2-butyl, tert-butyl.

Cycloalkyl radicals within the meaning of the invention are cyclic, saturated hydrocarbon radicals having 3 to 8 C atoms, preferably cyclopropyl, cyclobutyl, cyclopentyl, cyclohexyl.

Alkenyl radicals within the meaning of the invention are linear or branched, monounsaturated hydrocarbon radicals having 2 to 10, preferably 2 to 4 carbon atoms, preferably ethenyl, 1-propenyl, 2-propenyl, 1-buten-1-yl, 1-buten-2-yl, 2-buten-2-yl, 2-buten-1-yl, 3-buten-1-yl and 2-methyl-2-propen-1-yl.

Cycloalkenyl radicals within the meaning of the invention are cyclic, monounsaturated hydrocarbon radicals having 3 to 8, preferably 3 to 6 carbon atoms, preferably 1-cyclopropenyl, 2-cyclopropenyl, 1-cyclobutenyl, 2-cyclobutenyl, 1-cyclopentenyl, 2-cyclopentenyl.

### DETAILED DESCRIPTION OF THE PREFERRED EMBODIMENTS

Preferred for use as a fragrance and/or aroma substance are fatty acid esters or mixtures of two or more different fatty acid esters having formula (I), wherein for the, at least one, or each fatty acid ester having formula (I)
R¹ is an alkyl radical having 1 to 4 C atoms,
   a cycloalkyl radical having 3 to 6 C atoms,
   an alkenyl radical having 2 to 4 C atoms or
   a cycloalkenyl radical having 3 to 6 C atoms,
      and/or (preferably and)
R² is an alkyl radical having 1 to 3 C atoms
   and/or (preferably and)
n denotes 2, 3 or 4.

The above specifications for R¹, R² and n preferably apply simultaneously.

Most particularly preferred are fatty acid esters wherein
R¹ denotes an alkyl radical which is selected from the group consisting of methyl, ethyl, 1-propyl, 2-propyl, 1-butyl, 2-butyl and tert-butyl,
   a cycloalkyl radical which is selected from the group consisting of cyclopropyl, cyclobutyl, cyclopentyl and cyclohexyl,
   an alkenyl radical which is selected from the group consisting of ethenyl, 1-propenyl, 2-propenyl, 1-buten-1-yl, 1-buten-2-yl, 2-buten-2-yl, 2-buten-1-yl, 3-buten-1-yl and 2-methyl-2-propen-1-yl or
   a cycloalkenyl radical which is selected from the group consisting of 1-cyclopropenyl, 2-cyclopropenyl, 1-cyclobutenyl, 2-cyclobutenyl, 1-cyclopentenyl and 2-cyclopentenyl
   and/or (preferably and)
R² denotes an alkyl radical which is selected from the group consisting of methyl, ethyl and 1-propyl.

Here too the particularly preferred meanings of R¹ and R² preferably apply simultaneously.

In each case, the double bonds in the fatty acid radical of an alkadienoic acid ester having formula (I) are mutually independently (E)- or (Z)-configured.

Particularly preferred is the use (i) of a fatty acid ester having formula (I) or (ii) a mixture of two or more different fatty acid esters having the above formula (I), wherein the, at least one or each fatty acid ester having formula (I) is selected from the group consisting of:
2,7-nonadienoic acid methyl ester,
2,7-decadienoic acid methyl ester,
2,7-undecadienoic acid methyl ester,
2,8-decadienoic acid methyl ester,
2,8-undecadienoic acid methyl ester,
2,8-dodecadienoic acid methyl ester,
2,9-undecadienoic acid methyl ester,
2,9-dodecadienoic acid methyl ester,
2,9-tridecadienoic acid methyl ester,
2,7-nonadienoic acid ethyl ester,
2,7-decadienoic acid ethyl ester,
2,7-undecadienoic acid ethyl ester,
2,8-decadienoic acid ethyl ester,
2,8-undecadienoic acid ethyl ester,
2,8-dodecadienoic acid ethyl ester,
2,9-undecadienoic acid ethyl ester,
2,9-dodecadienoic acid ethyl ester,
2,9-tridecadienoic acid ethyl ester,
2,7-nonadienoic acid allyl ester,
2,7-decadienoic acid allyl ester,
2,7-undecadienoic acid allyl ester,
2,8-decadienoic acid allyl ester,
2,8-undecadienoic acid allyl ester,
2,8-dodecadienoic acid allyl ester,
2,9-undecadienoic acid allyl ester,
2,9-dodecadienoic acid allyl ester,
2,9-tridecadienoic acid allyl ester,
2,7-nonadienoic acid tert-butyl ester,
2,7-decadienoic acid tert-butyl ester,
2,7-undecadienoic acid tert-butyl ester,
2,8-decadienoic acid tert-butyl ester,
2,8-undecadienoic acid tert-butyl ester,
2,8-dodecadienoic acid tert-butyl ester,
2,9-undecadienoic acid tert-butyl ester
2,9-dodecadienoic acid tert-butyl ester and
2,9-tridecadienoic acid tert-butyl ester.

See also the appended Table 1 in this regard.

Particularly preferred alkadienoic acid esters having formula (I) are 2,7-decadienoic acid esters (R²=ethyl; n=2) and 2,8-undecadienoic acid esters (R²=ethyl; n=3) and mixtures thereof, wherein R¹ is further preferably an alkyl radical having 1 to 4 C atoms or an alkenyl radical having 2 to 4 C atoms.

In particularly preferred usages, the, at least one or each fatty acid ester having formula (I) is selected from the group consisting of:
2,7-decadienoic acid ethyl ester,
2,7-decadienoic acid methyl ester,
2,7-decadienoic acid tert-butyl ester,
2,7-decadienoic acid allyl ester,
2,8-undecadienoic acid ethyl ester,
2,8-undecadienoic acid methyl ester,
2,8-undecadienoic acid tert-butyl ester and
2,8-undecadienoic acid allyl ester.

By reason of their fragrance characteristics, it is advantageous if the, at least one or each fatty acid ester having formula (I) is selected from the group consisting of:
2,7-decadienoic acid ethyl ester,
2,7-decadienoic acid methyl ester,
2,8-undecadienoic acid ethyl ester and
2,8-undecadienoic acid methyl ester.

Particularly preferred in this regard are the (2E,7Z) and (2E,8Z) isomers of the cited 2,7-decadienoic acid esters and 2,8-undecadienoic acid esters or mixtures thereof in which the (2E,7Z) or (2E,8Z) isomers make up at least 80 mol %, more preferably at least 90 mol %, based on the total amount of alkadienoic acid esters having formula (I) that are used.

The very intense and interesting scent of the alkadienoic acid esters having formula (I), which is no longer pear-like but rather green, fruity, watery, floral and with aspects of melon, cucumber, apple, violet and/or lily-of-the-valley, in some cases aldehydic and somewhat fatty, was surprising and not foreseeable by the person skilled in the art. Furthermore, the alkadienoic acid esters having formula (I) exhibit good tenacity and are therefore particularly suitable for use in perfume oils and/or aroma compositions.

It has now been found in connection with the present invention that the cited alkadienoic acid esters having formula (I) (in particular those described as being (particularly) preferred) are extremely suitable for imparting, modifying and/or strengthening a scent having one or more of the notes green, fruity, watery, floral, aldehydic, fatty, melon, cucumber, apple, violet and/or lily-of-the-valley.

The cited alkadienoic acid esters having formula (I) (in particular those described above as being (particularly) preferred) are suitable in particular for being incorporated into perfume oils, especially for being incorporated into those having green, fruity, floral, aldehydic and/or watery notes. The alkadienoic acid esters having formula (I) can be used in a wide variety of perfume compositions, for example also in floral scent themes (see also example 5).

It has moreover been found that the cited alkadienoic acid esters having formula (I) (in particular those described above as being (particularly) preferred) are extremely suitable for imparting, modifying and/or strengthening a flavour having one or more of the notes juicy, green, cucumber, peel-like, melon, grape and/or aldehyde.

In particular, the alkadienoic acid esters having formula (I) (in particular those described above as being (particularly) preferred) are suitable for being incorporated into aroma compositions having green, fruity, juicy and/or watery notes, in particular notes of (water) melon and cucumber.

According to a further aspect of the invention, it was also found that compounds having formula (I), particularly those described above as being preferred, but in particular 2,8-undecadienoic acid esters having formula (I), and especially the (2E,8Z) isomers thereof, are extremely suitable for strengthening a scent or flavour, i.e. that the compounds having formula (I) can act as so-called boosters (strengtheners, enhancers). Therefore the invention also concerns the use of compounds having formula (I) as boosters (strengtheners, enhancers) for other fragrances or aroma substances, in particular for other fragrances or aroma substances having green, fresh, floral, fruity and/or aldehydic notes.

The present invention also concerns perfumed or aromatized products (in particular fragrance or aroma compositions and products containing a fragrance or aroma composition), processes for imparting, modifying and/or strengthening certain scent and/or flavour notes and new compounds having formula (I).

Among the alkadienoic acid esters having formula (I), only a few are already known, which—taken by themselves—are not provided by the present invention.

The 2,8-undecadienoic acid esters having formula (I) are novel.

Page 895 of Helv. Chim. Acta, 1978, 885-898 discloses the compounds (2E,7E)- and (2E,7Z)-decadienoic acid methyl ester and the compound (2E,7Z)-decadienoic acid ethyl ester. It states in particular that 10 mmol of triethylphosphonoacetic acid ethyl and methyl esters in 5 ml of benzene were reacted with 10 mmol of NaH dispersion (washed free from mineral oil) for one hour at room temperature. 10 mmol of 5-octenal in 5 ml of benzene were added dropwise to the clear ylide solution at room temperature. A viscous deposit formed. Stirring was then continued for 5 hours at 60-70°, the mixture was cooled, the deposit filtered off, washed out with warm benzene, the filtrates were evaporated and the residue distilled in a bulb tube. (2E,7E)-decadienoic acid methyl ester and (2E,7Z)-decadienoic acid ethyl ester respectively were obtained.

Said page of the cited document further discloses that the ethyl ester was transesterified with 2 N HCl in methanol at room temperature for 40 hours to produce (2E,7Z)-decadienoic acid methyl ester.

Said page of the cited document also discloses that the methyl esters were able to be separated off under certain conditions, mention being made in particular of the injection of an ethereal solution.

Finally said page of the cited document also discloses that 1 mmol of the decadienoic acid methyl ester in 5 ml of benzene was reacted with 1 mmol of m-chloroperbenzoic acid.

From the above details of the disclosure of the cited publication and from a study of the publication itself, it emerges that (2E,7E)- and (2E,7Z)-decadienoic acid methyl ester and (2E,7Z)-decadienoic acid ethyl ester were used only as synthesis intermediates in the context of the publication. The use according to the invention of 2,7-decadienoic acid methyl and ethyl esters as a fragrance and aroma substance is not disclosed in Helv. Chim. Acta, 1978, 885-898.

All in all the use of 2,7-decadienoic acid esters having formula (I) as a fragrance or aroma substance or their corresponding use in products is not known from the prior art.

The alkadienoic acid esters having formula (I) can be produced by reacting an aldehyde having formula (II) 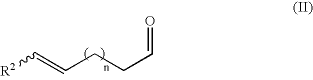
wherein R² and n have the above meaning (preferably a meaning described above as being preferred)
with
a phosphono ester having the general formula (III) 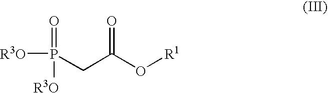
wherein R¹ has the above meaning (preferably a meaning described above as being preferred),
and each R³ is a linear or branched alkyl radical, preferably an alkyl radical having 1 to 10, preferably 1 to 4 C atoms, preferably methyl, ethyl, 1-propyl, 2-propyl, 1-butyl, 2-butyl, tert-butyl.

Conventionally, the reaction mixture is processed, and the alkadienoic acid esters having formula (I) that are formed are optionally purified.

The reaction is generally performed in the presence of an auxiliary base and a solvent or a solvent blend, both aqueous and non-aqueous systems being able to be used, preferably with the aid of a phase-transfer catalyst.

The reaction is particularly preferably performed in the presence of water, a weak base, preferably potassium hydrogen carbonate or potassium carbonate, and a phase-transfer catalyst, preferably a quaternary ammonium salt, e.g. tetrabutyl ammonium bromide or cetyl trimethyl ammonium chloride, being used and the reaction being performed at temperatures of 0 to 100° C., preferably 10 to 80° C.

In a particularly preferred embodiment, for the purposes of processing and purification, at the end of the reaction, optionally after the addition of a lipophilic solvent, preferably a hydrocarbon, the mixture is simply split into a lipophilic and an aqueous phase, the lipophilic mixture is washed with water and then undergoes a gentle fractionating distillation under vacuum, high yields of the preferred main isomer being obtained in fragrance quality.

As mentioned, the present invention also concerns perfumed or aromatized products, including preparations, semi-finished products and fragrance, aroma and flavouring compositions.

Perfumed or aromatized products according to the invention include:
(a) (i) a fatty acid ester having formula (I) or
   (ii) a mixture of two or more different fatty acid esters having formula (I) 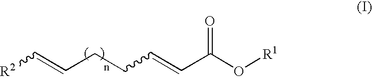
      wherein
R¹ is an alkyl radical having 1 to 10 C atoms,
   a cycloalkyl radical having 3 to 8 C atoms,
   an alkenyl radical having 2 to 10 C atoms or
   a cycloalkenyl radical having 3 to 8 C atoms,
R² is an alkyl radical having 1 to 10 C atoms,
n denotes 1, 2, 3, 4 or 5,
   and
   the two double bonds in the fatty acid radical are mutually independently (E)- or (Z)-configured,
   with the proviso that the product
   (i) is not a product (in particular a composition) containing (2E,7E)- or (2E,7Z)-decadienoic acid methyl ester or (2E,7Z)-decadienoic acid ethyl ester and other constituents as described on page 895 of Helv. Chim. Acta, 1978, 885-898 (in particular in the passages mentioned above)
   and/or
   (ii) does not contain benzene
   and/or
   (iii) does not contain 5-octenal
   and/or
   (iv) does not contain methanol
      and
(b) one, two, three or more other fragrances or aroma substances.

Preferred perfumed or aromatized products (including preparations) according to the invention are in particular cosmetic, body care, household, food, consumable or oral hygiene (including oral care) preparations and dermatological or (oral) pharmaceutical preparations.

It is advantageous to combine one or more alkadienoic acid esters having formula (I) with at least one further fragrance or aroma substance and thus to form a novel fragrance or aroma composition (as an example of a product according to the invention). Particularly interesting and natural, novel and original fragrance notes can be created in this way. Fragrances which are advantageously suitable for combining can be found for example in S. Arctander, Perfume and Flavor Materials, Vol. I and II, Montclair, N.J. 1969, self-published, or K. Bauer et al., Common Fragrance and Flavor Materials, 4th Edition, Wiley-VCH, Weinheim 2001. The following specific examples can be cited:

Extracts of natural raw materials such as essential oils, concretes, absolutes, resins, resinoids, balsams, tinctures, such as, for example,

ambergris tincture; amyris oil; angelica seed oil; angelica root oil; aniseed oil; valerian oil; basil oil; treemoss absolute; bay oil; mugwort oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute; cedar leaf oil; cedarwood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oakmoss absolute; elemi oil; tarragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camomile blue oil; camomile Roman oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; litsea cubeba oil; bayleaf oil; mace oil; marjoram oil; mandarin oil; massoi bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange flower absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir needle oil; tea-tree oil; turpentine oil; thyme oil; tolu balsam; tonka absolute; tuberose absolute; vanilla extract; violet leaf absolute; verbena oil; vetiver oil; juniper berry oil; cognac oil; wormwood oil; wintergreen oil; ylang-ylang oil; hyssop oil; civet absolute; cinnamon leaf oil; cinnamon bark oil and fractions thereof, or ingredients isolated therefrom;

individual fragrances from the group of hydrocarbons, such as, for example, 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenyl methane;

aliphatic alcohols, such as, for example, hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methylene heptan-2-ol; (E,Z)-2,6-nonadienol; 3,7-dimethyl-7-methoxyoctan-2-ol; 9-decenol; 10-undecenol; 4-methyl-3-decen-5-ol;

aliphatic aldehydes and acetals thereof, such as, for example, hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethyl acetal; 1,1-dimethoxy-2,2,5-trimethyl-4-hexene; citronellyl oxyacetaldehyde; 1-(1-methoxypropoxy)-(E/Z)-3-hexene; aliphatic ketones and oximes thereof, such as, for example, 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;

aliphatic sulfur-containing compounds, such as, for example, 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

aliphatic nitriles, such as, for example, 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;

esters of aliphatic carboxylic acids, such as, for example, (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl-2-methylpentanoate; ethyl hexanoate; allyl hexanoate; ethyl heptanoate; allyl heptanoate; ethyl octanoate; ethyl-(E,Z)-2,4-decadienoate; methyl-2-octynoate; methyl-2-nonynoate; allyl-2-isoamyloxyacetate; methyl-3,7-dimethyl-2,6-octadienoate; 4-methyl-2-pentyl crotonate;

acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates and 3-methyl-2-butenoates thereof;

acyclic terpene aldehydes and ketones, such as, for example, geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;

cyclic terpene alcohols, such as, for example, menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and formates, acetates, propionates, isobutyrates, butyrates, isovalerates, pentanoates, hexanoates, crotonates, tiglinates and 3-methyl-2-butenoates thereof;

cyclic terpene aldehydes and ketones, such as, for example, menthone; isomenthone; 8-mercaptomenthan-3-one; carvone; camphor; fenchone; alphaionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4-a-methanonaphthalen-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methyl cedryl ketone);

cyclic alcohols, such as, for example, 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

cycloaliphatic alcohols, such as, for example, alpha-3,3-trimethyl cyclohexylmethanol; 1-(4-isopropylcyclohexyl)ethanol; 2-methyl-4-(2,2,3-trimethyl-3-cyclopent-1-yl) butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

cyclic and cycloaliphatic ethers, such as, for example, cineol; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methyl propyl)-1,3-dioxane;

cyclic and macrocyclic ketones, such as, for example, 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;

cycloaliphatic aldehydes such as, for example, 2,4-dimethyl-3-cyclohexene carbaldehyde; 2-methyl-4-(2,2,6-trimethyl cyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde; esters of cyclic alcohols such as, for example, 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;

esters of cycloaliphatic alcohols such as, for example, 1-cyclohexylethyl crotonate;

esters of cycloaliphatic carboxylic acids, such as, for example, allyl-3-cyclohexyl propionate; allyl cyclohexyloxyacetate; cis- and trans-methyl dihydrojasmonate; cis- and trans-methyl jasmonate; methyl-2-hexyl-3-oxocyclopentanecarboxylate; ethyl-2-ethyl-6,6-dimethyl-2-cyclohexenecarboxylate; ethyl-2,3,6,6-tetramethyl-2-cyclohexenecarboxylate; ethyl-2-methyl-1,3-dioxolane-2-acetate;

araliphatic alcohols, such as, for example, benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;

araliphatic ethers, such as, for example, 2-phenylethyl methyl ether; 2-phenylethyl isoamyl ether; 2-phenylethyl-1-ethoxyethyl ether; phenylacetaldehyde dimethyl acetal; phenylacetaldehyde diethyl acetal; hydratropaldehyde dimethyl acetal;

phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

aromatic and araliphatic aldehydes, such as, for example, benzaldehyde; phenylacetaldehyde; 3-phenylpropanal; hydratropic aldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tertbutylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

aromatic and araliphatic ketones, such as, for example, acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranyl ethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

aromatic and araliphatic carboxylic acids and esters thereof, such as, for example, benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl-2,4-dihydroxy-3,6-dimethylbenzoate; ethyl-3-phenylglycidate; ethyl-3-methyl-3-phenylglycidate;

nitrogen-containing aromatic compounds, such as, for example, 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenoic acid nitrile; 3-methyl-5-phenyl pentanoic acid nitrile; methyl anthranilate; methyl-N-methyl anthranilate; Schiff bases of methyl anthranilate with 7-hydroxy-3,7-dimethyloctanal; 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;

phenols, phenyl ethers and phenyl esters, such as, for example, estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

heterocyclic compounds, such as, for example, 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene-1,12-dodecanedioate; ethylene-1,13-tridecanedioate; coumarin; 2,3-dihydrocoumarin; octahydrocoumarin.

The total amount of the one or more alkadienoic acid esters having formula (I) used in fragrance or aroma compositions according to the invention is preferably 0.00001 to 90 wt. %, preferably 0.001 to 70 wt. %, and particularly preferably 0.01 to 50 wt. %, based on the total amount of fragrance or aroma composition.

In fragrance or aroma compositions according to the invention the ratio by weight of the total amount of fatty acid esters having formula (I) to the total amount of further fragrances or aroma substances is in the range from 1:1000 to 1:0.5. Fragrance or aroma compositions according to the invention containing one or more alkadienoic acid esters having formula (I) can be used in liquid form, undiluted or diluted with a solvent, for perfuming or aromatizing. Suitable solvents for this purpose are, for example, ethanol, isopropanol, diethylene glycol monoethyl ether, glycerol, propylene glycol, 1,2-butylene glycol, dipropylene glycol, diethyl phthalate, triethyl citrate, isopropyl myristate, triacetin, etc.

Benzene and methanol are generally not a constituent of a fragrance or aroma composition according to the invention.

Fragrance or aroma compositions according to the invention containing one or more alkadienoic acid esters having formula (I) can also be adsorbed on a carrier, which ensures both a fine distribution of the fragrances or aroma substances in the product and a controlled release during use. Such carriers can be porous inorganic materials such as light sulfate, silica gels, zeolites, gypsums, clays, clay granules, gas concrete, etc., or organic materials such as woods, cellulose-based substances, sugars, dextrins (e.g. maltodextrin) or plastics such as PVC, polyvinyl acetates or polyurethanes. The combination of composition according to the invention and carrier is an example of a product according to the invention.

Fragrance or aroma compositions according to the invention containing one or more alkadienoic acid esters having formula (I) can also be used in microencapsulated or spray-dried form, in the form of inclusion complexes or extrusion products (i.e. products according to the invention) and added in this form to a product to be perfumed or aromatized, for example.

A preferred perfumed or aromatized product according to the invention contains a total amount of fatty acid esters having formula (I) which is sufficient to impart, to modify and/or to strengthen a
scent note of the type green, fruity, watery, floral, aldehydic, fatty, melon, cucumber, apple, violet and/or lily-of-the-valley
   or a
flavour note of the type juicy, green, cucumber, peel-like, melon, grape and/or aldehyde.

Such a product according to the invention is preferably a fragrance or aroma composition.

The properties of the compositions modified in such a way can optionally be further optimised by coating with suitable materials with in view of a more selective fragrance release, waxy plastics such as e.g. polyvinyl alcohol being preferably used. The resulting products are likewise products according to the invention.

Microencapsulation of the fragrance or aroma compositions according to the invention to form products according to the invention can take place by means of the so-called coacervation process, for example, using capsule materials made from polyurethane-like substances or soft gelatine, for example. The spray-dried fragrance or aroma compositions can be prepared by spray drying an emulsion or dispersion containing the fragrance or aroma composition, for example, wherein modified starches, proteins, dextrin and vegetable gums can be used as carriers. Inclusion complexes can be prepared by, for example, introducing dispersions of the fragrance or aroma composition and cyclodextrins or urea derivatives into a suitable solvent, e.g. water. Extrusion products can be obtained by melting the fragrance or aroma compositions with a suitable waxy substance and by extrusion with subsequent solidification, optionally in a suitable solvent, e.g. isopropanol.

The alkadienoic acid esters having formula (I) for use according to the invention and fragrance compositions containing one or more such alkadienoic acid esters having formula (I) can be used in concentrated form, in solutions or in the aforementioned modified form for the production of perfumed products according to the invention such as e.g. perfume extracts, eau de parfums, eau de toilettes, aftershaves, eau de colognes, preshave products, splash colognes and perfumed refreshing wipes, and for the perfuming of acid, alkaline and neutral cleaning agents, such as e.g. floor cleaners, window cleaners, washing-up liquids, bath and sanitary cleaners, scouring agent, solid and liquid WC cleaners, powdered and foaming carpet cleaners, textile fresheners, ironing aids, liquid detergents, powdered detergents, laundry pretreatment agents such as bleaches, soaking agents and stain removers, fabric softeners, laundry soaps, detergent tablets, disinfectants, surface disinfectants and air fresheners in liquid or gel form or applied to a solid support, aerosol sprays, waxes and polishes such as furniture polishes, floor waxes, shoe creams and personal care products such as e.g. solid and liquid soaps, shower gels, shampoos, shaving soaps, shaving foams, bath oils, cosmetic emulsions of the oil-in-water, water-in-oil and water-in-oil-in-water type, such as e.g. skin creams and lotions, face creams and lotions, sun creams and lotions, aftersun creams and lotions, hand creams and lotions, foot creams and lotions, depilatory creams and lotions, aftershave creams and lotions, tanning creams and lotions, haircare products such as e.g. hair sprays, hair gels, hair setting lotions, hair rinses, permanent and semi-permanent hair colorants, hair sculpting agents such as cold waves and hair smoothing agents, hair waters, hair creams and lotions, deodorants and antiperspirants such as e.g. underarm sprays, roll-ons, deodorant sticks, deodorant creams, decorative cosmetic products such as e.g. eyeshadows, nail polishes, foundations, lipsticks, mascara, and of candles, lamp oils, incense sticks, insecticides, repellents and fuels.

The alkadienoic acid esters having formula (I) and mixtures thereof for use according to the invention can be incorporated into aromatized products or products to be aromatized, in particular food, oral care or consumable preparations.

Food or consumable preparations are, for example, baked goods (e.g. bread, dry biscuits, cakes, other pastries), confectionery (e.g. chocolates, chocolate bar products, other bar products, fruit gums, hard-boiled and soft sweets, chewing gum), alcoholic or non-alcoholic drinks (e.g. coffee, tea, wine, wine-based drinks, beer, beer-based drinks, liqueurs, spirits, brandies, fruit-based soft drinks, isotonic drinks, soft drinks, nectars, fruit and vegetable juices, fruit or vegetable juice preparations), instant drinks (e.g. instant chocolate drinks, instant tea drinks, instant coffee drinks), meat products (e.g. ham, cured or uncured sausage preparations, spiced or marinated fresh or salted meat products), eggs or egg products (dried egg, egg white, egg yolk), cereal products (e.g. breakfast cereals, muesli bars, pre-cooked convenience rice products), dairy products (e.g. milk drinks, ice cream, yoghurt, kefir, cream cheese, soft cheese, hard cheese, dried milk powder, whey, butter, buttermilk, partially or completely hydrolysed milk protein-containing products), products made from soya protein or other soya bean fractions (e.g. soya milk and products made therefrom, preparations containing soya lecithin, fermented products such as tofu or tempe or products made therefrom, soy sauces), fruit preparations (e.g. jams, fruit sorbets, fruit sauces, fruit fillings), vegetable preparations (e.g. ketchup, sauces, dried vegetables, frozen vegetables, pre-cooked vegetables, pickled vegetables, preserved vegetables), snacks (e.g. baked or fried potato crisps or potato dough products, bread dough products, extruded products based on maize or peanuts), products based on fats and oils or emulsions thereof (e.g. mayonnaise, remoulade, dressings, condiment preparations), other ready meals and soups (e.g. dried soups, instant soups, pre-cooked soups), spices, spice mixes and in particular seasonings, which are used in the snacks sector for example. After incorporation of the alkadienoic acid esters having formula (I) for use according to the invention, these preparations are preparations according to the invention.

Preparations according to the invention can also take the form of a semi-finished product or a spice mix.

Preparations according to the invention can be used in particular as semi-finished products for the production of other food or consumable preparations, in particular in spray-dried form. Preparations according to the invention can also take the form of capsules, tablets (uncoated and coated tablets, e.g. stomach acid-resistant coatings), pastilles, granules, pellets, solids mixtures, dispersions in liquid phases, emulsions, powders, solutions, pastes or other swallowable or chewable preparations as food supplements.

Oral care preparations according to the invention are in particular oral and/or dental care products such as toothpastes, tooth gels, tooth powders, dental floss, toothpicks, mouthwashes and chewing gums.

Other conventional active ingredients, basic substances, auxiliary substances and additives for food, oral care or consumable preparations according to the invention can be included in amounts of 5 to 99.99 wt. %, preferably 10 to 80 wt. %, based on the total weight of the preparation. The preparations can also contain water in an amount of up to 99.99 wt. %, preferably 5 to 80 wt. %, based on the total weight of the preparation.

The preparations according to the invention (as examples of products according to the invention) containing one or more alkadienoic acid esters having formula (I) are produced according to a preferred embodiment by incorporating one or more alkadienoic acid esters having formula (I) in bulk, as a solution (e.g. in ethanol, water or 1,2-propylene glycol) or in the form of a mixture with a solid or liquid carrier (e.g. maltodextrin, starch, silica gel), other aromas or aroma substances and optionally other auxiliary substances and/or stabilisers (e.g. natural or synthetic polysaccharides and/or vegetable gums such as modified starches or gum arabic) into a basic preparation for food, oral care or consumption. Preparations according to the invention in the form of a solution and/or suspension or emulsion can advantageously also be converted to a solid preparation according to the invention (semi-finished product) by spray drying.

As semi-finished products, the spray-dried solid preparations according to the invention (as an example of products according to the invention) are particularly suitable for the production of further preparations according to the invention. The spray-dried solid preparations according to the invention preferably contain 50 to 95 wt. % of carriers, in particular maltodextrin and/or starch, 5 to 40% of auxiliary substances, preferably natural or synthetic polysaccharides and/or vegetable gums such as modified starches or gum arabic.

According to a further preferred embodiment, one or more alkadienoic acid esters having formula (I) and optionally other constituents of the preparation according to the invention are first incorporated into emulsions, liposomes, e.g. starting from phosphatidyl cholin, microspheres, nanospheres or into capsules, granules or extruded products from a suitable matrix for foodstuffs and consumable products, e.g. from starch, starch derivatives (e.g. modified starch), cellulose or cellulose derivatives (e.g. hydroxypropyl cellulose), other polysaccharides (e.g. dextrin, alginate, curdlan, carageenan, chitin, chitosan, pullulan), natural fats, natural waxes (e.g. beeswax, carnauba wax) from proteins, e.g. gelatine, or other natural products (e.g. shellac) to produce preparations according to the invention. Depending on the matrix, the products can be obtained by spray drying, spray granulation, melt granulation, coacervation, coagulation, extrusion, melt extrusion, emulsion processes, coating or other suitable encapsulation processes and optionally a suitable combination of the above processes. In a further preferred production process for a preparation according to the invention, the alkadienoic acid ester(s) having formula (I) are first complexed with one or more suitable complexing agents, for example with cyclodextrins or cyclodextrin derivatives, preferably alpha- or beta-cyclodextrin, and used in this complexed form.

A preparation according to the invention is particularly preferred wherein the matrix is chosen such that the alkadienoic acid esters having formula (I) undergo a delayed release from the matrix, such that a lasting effect is achieved. A fat, wax, polysaccharide or protein matrix is particularly preferred in this respect.

As other constituents for food or consumable preparations according to the invention, conventional basic substances, auxiliary substances and additives for foodstuffs or consumables can be used, e.g. water, mixtures of fresh or processed, plant-based or animal-based basic substances or raw materials (e.g. raw, roast, dried, fermented, smoked and/or boiled meat, bone, gristle, fish, vegetables, fruit, herbs, nuts, vegetable or fruit juices or pastes or mixtures thereof), digestible or indigestible carbohydrates (e.g. sucrose, maltose, fructose, glucose, dextrin, amylose, amylopectin, inulin, xylan, cellulose, tagatose), sugar alcohols (e.g. sorbitol, erythritol), natural or hydrogenated fats (e.g. tallow, lard, palm oil, coconut butter, hydrogenated vegetable fat), oils (e.g. sunflower oil, groundnut oil, maize oil, olive oil, fish oil, soya oil, sesame oil), fatty acids or salts thereof (e.g. potassium stearate), proteinogenic or non-proteinogenic amino acids and related compounds (e.g. γ-aminobutyric acid, taurin), peptides (e.g. glutathione), native or processed proteins (e.g. gelatine), enzymes (e.g. peptidases), nucleic acids, nucleotides, flavour correctors for unpleasant flavour impressions, other flavour modulators for other, generally not unpleasant flavour impressions, other flavour-modulating substances (e.g. inositol phosphate, nucleotides such as guanosine monophosphate, adenosine monophosphate or other substances such as sodium glutamate or 2-phenoxypropionic acid), emulsifiers (e.g. lecithins, diacyl glycerols, gum arabic), stabilisers (e.g. carageenan, alginate), preservatives (e.g. benzoic acid, sorbic acid), antioxidants (e.g. tocopherol, ascorbic acid), chelating agents (e.g. citric acid), organic or inorganic acidulators (e.g. malic acid, acetic acid, citric acid, tannic acid, phosphoric acid), bitter principles (e.g. quinine, caffeine, limonin, amarogentin, humolones, lupolones, catechins, tannins), mineral salts (e.g. sodium chloride, potassium chloride, magnesium chloride, sodium phosphates), substances preventing enzymatic browning (e.g. sulfite, ascorbic acid), essential oils, plant extracts, natural or synthetic dyes or coloured pigments (e.g. carotenoids, flavonoids, anthocyans, chlorophyll and derivatives thereof), herbs, trigeminally active substances or plant extracts containing such trigeminally active substances, synthetic, natural or nature-identical aroma substances or fragrances and odour correctors.

Dental care products according to the invention (as a basis for oral care preparations) which contain one or more alkadienoic acid esters having formula (I) generally contain an abrasive system (grinding or polishing agent), such as e.g. silicas, calcium carbonates, calcium phosphates, aluminium oxides and/or hydroxyl apatites, surface-active substances, such as e.g. sodium lauryl sulfate, sodium lauryl sarcosinate and/or cocamidopropyl betaine, humectants, such as e.g. glycerol and/or sorbitol, thickeners, such as e.g. carboxymethyl cellulose, polyethylene glycols, carrageenan and/or Laponite®, sweeteners, such as e.g. saccharine, flavour correctors for unpleasant flavour impressions, flavour correctors for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), cooling agents, such as e.g. menthol or menthol derivatives (e.g. L-menthol, L-menthyl lactate, L-menthylalkyl carbonates, menthone ketals, menthane carboxylic acid amides), 2,2,2-trialkyl acetic acid amides (e.g. 2,2-diisopropyl propionic acid methyl amide), icilin and icilin derivatives, stabilisers and active ingredients, such as e.g. sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetyl pyridinium chloride, aluminium lactate, potassium citrate, potassium nitrate, potassium chloride, strontium chloride, hydrogen peroxide, aromas and/or sodium bicarbonate or odour correctors.

Chewing gums according to the invention (as a further example of oral care preparations), which contain one or more alkadienoic acid esters having formula (I), generally comprise a chewing gum base, in other words a chewing compound that becomes plastic when chewed, sugars of various types, sugar substitutes, other sweet-tasting substances, sugar alcohols, flavour correctors for unpleasant flavour impressions, other flavour modulators for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), humectants, thickeners, emulsifiers, aromas and stabilisers or odour correctors.

The present invention also concerns a corresponding process for imparting, modifying and/or strengthening a
scent note of the type green, fruity, watery, floral, aldehydic, fatty, melon, cucumber, apple, violet and/or lily-of-the-valley
   or a
flavour note of the type juicy, green, cucumber, peel-like, melon, grape and/or aldehyde,
   wherein a sensorially effective amount of a fragrance or aroma composition according to the invention or of one or more fatty acid esters for use according to the invention is brought into contact with or mixed with a product.

With regard to preferred embodiments of the process according to the invention, the comments made regarding the use according to the invention apply accordingly.

As already mentioned, the present invention also concerns novel fatty acid esters having formula (I). The present invention thus concerns in very general terms fatty acid esters having formula (I) 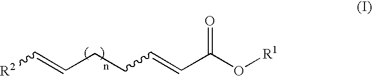
wherein
R¹ is an alkyl radical having 1 to 10 C atoms,
   a cycloalkyl radical having 3 to 8 C atoms,
   an alkenyl radical having 2 to 10 C atoms or
   a cycloalkenyl radical having 3 to 8 C atoms,
R² is an alkyl radical having 1 to 10 C atoms,
n denotes 1, 2, 3, 4 or 5,
   and
   the two double bonds in the fatty acid radical are mutually independently (E)- or (Z)-configured,
   with the exception of (2E,7E)- and (2E,7Z)-decadienoic acid methyl ester and (2E,7Z)-decadienoic acid ethyl ester.

Preferred fatty acid esters having formula (I) according to the invention are those which (i) contain at most 18 C atoms and at least 9 C atoms. However, the fatty acid esters having formula (I) preferably contain (ii) at most 16 C atoms and at least 9 C atoms and they most particularly preferably contain (iii) at most 16 C atoms and at least 10 C atoms.

As similarly already described for the use according to the invention, fatty acid esters are preferred wherein in formula (I)
R¹ is an alkyl radical having 1 to 4 C atoms,
   a cycloalkyl radical having 3 to 6 C atoms,
   an alkenyl radical having 2 to 4 C atoms or
   a cycloalkenyl radical having 3 to 6 C atoms,
      and/or (preferably and)
R² is an alkyl radical having 1 to 3 C atoms
   and/or (preferably and)
n denotes 2, 3 or 4.

Most particularly preferred are fatty acid esters having formula (I) wherein
R¹ denotes an alkyl radical which is selected from the group consisting of methyl, ethyl, 1-propyl, 2-propyl, 1-butyl, 2-butyl and tert-butyl,
   a cycloalkyl radical which is selected from the group consisting of cyclopropyl, cyclobutyl, cyclopentyl and cyclohexyl,
   an alkenyl radical which is selected from the group consisting of ethenyl, 1-propenyl, 2-propenyl, 1-buten-1-yl, 1-buten-2-yl, 2-buten-2-yl, 2-buten-1-yl, 3-buten-1-yl and 2-methyl-2-propen-1-yl
   or
   a cycloalkenyl radical which is selected from the group consisting of 1-cyclopropenyl, 2-cyclopropenyl, 1-cyclobutenyl, 2-cyclobutenyl, 1-cyclopentenyl and 2-cyclopentenyl
      and/or (preferably and)
R² denotes an alkyl radical which is selected from the group consisting of methyl, ethyl and 1-propyl.

As already described in connection with the uses according to the invention, in fatty acid esters according to the invention the specific meanings given for R¹, R² and n preferably apply simultaneously.

Particularly preferred novel fatty acid esters according to the invention are selected from the group consisting of:
2,7-nonadienoic acid methyl ester,
2,7-undecadienoic acid methyl ester,
2,8-decadienoic acid methyl ester,
2,8-undecadienoic acid methyl ester,
2,8-dodecadienoic acid methyl ester,
2,9-undecadienoic acid methyl ester,
2,9-dodecadienoic acid methyl ester,
2,9-tridecadienoic acid methyl ester,
2,7-nonadienoic acid ethyl ester,
2Z,7E-decadienoic acid ethyl ester,
2,7-undecadienoic acid ethyl ester,
2,8-decadienoic acid ethyl ester,
2,8-undecadienoic acid ethyl ester,
2,8-dodecadienoic acid ethyl ester,
2,9-undecadienoic acid ethyl ester,
2,9-dodecadienoic acid ethyl ester,
2,9-tridecadienoic acid ethyl ester,
2,7-nonadienoic acid allyl ester,
2,7-decadienoic acid allyl ester,
2,7-undecadienoic acid allyl ester,
2,8-decadienoic acid allyl ester,
2,8-undecadienoic acid allyl ester,
2,8-dodecadienoic acid allyl ester,
2,9-undecadienoic acid allyl ester,
2,9-dodecadienoic acid allyl ester,
2,9-tridecadienoic acid allyl ester,
2,7-nonadienoic acid tert-butyl ester,
2,7-decadienoic acid tert-butyl ester,
2,7-undecadienoic acid tert-butyl ester,
2,8-decadienoic acid tert-butyl ester,
2,8-undecadienoic acid tert-butyl ester,
2,8-dodecadienoic acid tert-butyl ester,
2,9-undecadienoic acid tert-butyl ester
2,9-dodecadienoic acid tert-butyl ester and
2,9-tridecadienoic acid tert-butyl ester.

Of these fatty acid esters, those which are particularly preferred are selected from the group consisting of:
2Z,7E-decadienoic acid ethyl ester,
2,7-decadienoic acid tert-butyl ester,
2,7-decadienoic acid allyl ester,
2,8-undecadienoic acid ethyl ester,
2,8-undecadienoic acid methyl ester,
2,8-undecadienoic acid tert-butyl ester and
2,8-undecadienoic acid allyl ester.

If no details are given above and below of the configuration at the two double bonds of the fatty acid radical of a fatty acid ester according to the invention, the configuration can be (E) or (Z), regardless of the configuration at a further double bond present in the molecule. It has already been stated above, however, that the (2E,7Z) and (2E,8Z) isomers of 2,7-decadienoic acid esters and 2,8-undecadienoic acid esters or mixtures thereof in which the (2E,7Z) or (2E,8Z) isomers make up at least 80 mol %, more preferably at least 90 mol %, based on the total amount of alkadienoic acid esters having formula (I) that are used, are preferred for the use according to the invention.

Of the novel fatty acid esters having formula (I), those which are most preferred are selected from the group consisting of:
2Z,7E-decadienoic acid ethyl ester,
2,8-undecadienoic acid ethyl ester (in particular (2E,8Z)) and
2,8-undecadienoic acid methyl ester (in particular (2E,8Z)).

Further aspects of the present invention follow from the appended claims and the description below. The invention is described below in more detail with reference to examples. Unless otherwise specified, all figures stated relate to the weight.

### Example 1

### (2E,7Z)-Decadienoic acid ethyl ester

### Prevailing Isomer

Aqueous potassium carbonate solution (50 ml, 50% (w/w)) and aqueous tributyl ammonium bromide solution (1.5 ml, 50% (w/w)) were measured out and triethyl phosphonoacetate (19.9 g, 88.8 mmol) was added. 5Z-Octenal (9.92 g, 78.6 mmol) was added over 5 minutes, causing the temperature of the mixture to rise to approx. 40° C. The reaction mixture was stirred for 16 h, then n-hexane was added, the mixture was separated after being thoroughly mixed together and the organic phase underwent fractionating distillation under vacuum. Yield 10.5 g colourless oil (82% yield, content of main isomer (2E,7Z): 94%, GC; d²⁵=0.9004; n_{D}²⁵=1.4596).

¹H-NMR (400 MHz, CDCl₃): δ=0.96 (3H, t, J=7.5 Hz, H-10), 1.28 (3H, t, J=7.2 Hz, H-2′), 1.52 (2H, tt, J=7.4 Hz, J=7.4 Hz, H-5), 2.03 (2H, m, J=7.6 Hz, H-6 or H-9), 2.06 (2H, m, J=7.3 Hz, H-9 or H-6), 2.21 (2H, tdd, J=8.0 Hz, J=7.0 Hz, J=1.6 Hz, H-4), 4.18 (2H, q, J=7.2 Hz, H-1′), 5.30 (1H, dtt, J=10.9 Hz, J=7.2 Hz, J=1.4 Hz, H-7), 5.40 (1H, dtt, J=10.9 Hz, J=7.1 Hz, J=1.4 Hz, H-8), 5.82 (1H, dt, J=15.7 Hz, J=1.6 Hz, H-2), 6.96 (1H, dt, J=15.6 Hz, J=7.0 Hz, H-3) ppm.

¹³C-NMR (100 MHz, CDCl₃): δ=14.30 (CH₃, C-10 or C-2′), 14.33 (CH₃, C2′ or C-10), 20.58 (CH₂, C-9), 26.52 (CH₂, C-6), 28.08 (CH₂, C-5), 31.70 (CH₂, C-4), 60.14 (CH₂, C-1′), 121.49 (CH, C-2), 128.14 (CH, C-7), 132.49 (CH, C-8), 149.10 (CH, C-3), 166.74 (C, C-1) ppm.

Scent profile (0.5 wt. % in diethyl phthalate): green, fruity (apple, melon), watery, violet, fatty

Flavour profile (2 ppm in aqueous 5% sucrose or 0.5% sodium chloride solution): juicy, green, cucumber, peel-like, melon, grape, sweet, tea, aldehyde

### Example 2

### (2E,8Z)-Undecadienoic acid ethyl ester

### Prevailing Isomer

Aqueous potassium carbonate solution (108 ml, 50% (w/w)) and aqueous tributyl ammonium bromide solution (3 ml, 50% (w/w)) were measured out and triethyl phosphonoacetate (40.3 g, 180 mmol) was added. 6Z-Nonenal (22.5 g, 160.5 mmol) was added over 5 minutes, causing the temperature of the mixture to rise to approx. 45° C. The reaction mixture was stirred for 16 h, then n-hexane was added, the mixture was separated after being thoroughly mixed together and the organic phase underwent fractionating distillation under vacuum. Yield 25.9 g colourless oil (92% yield, content of main isomer (2E,8Z): 86%, GC; d²⁵=0.8988; n_{D}²⁵=1.4603).

¹H-NMR (400 MHz, CDCl₃): δ=0.96 (3H, t, J=7.5 Hz, H-11), 1.28 (3H, t, J=7.2 Hz, H-2′), 1.38 (2H, m, H-6), 1.47 (2H, m, H-5), 2.03 (2H, m, H-7 or H-10), 2.04 (2H, m, H-10 or H-7), 2.20 (2H, tdd, J=7.1 Hz, J=7.1 Hz, J=1.6 Hz, H-4), 4.18 (2H, q, J=7.1 Hz, H-1′), 5.30 (1H, dtt, J=10.8 Hz, J=7.1 Hz, J=1.4 Hz, H-8), 5.37 (1H, dtt, J=10.8 Hz, J=7.1 Hz, J=1.4 Hz, H-9), 5.88 (1H, dt, J=15.6 Hz, J=1.6 Hz, H-2), 6.96 (1H, dt, J=15.6 Hz, J=7.0 Hz, H-3) ppm.

¹³C-NMR (100 MHz, CDCl₃): δ=14.30 (CH₃, C-11 or C-2′), 14.37 (CH₃, C2′ or C-11), 20.55 (CH₂, C-10), 26.80 (CH₂, C-7), 27.61 (CH₂, C-5), 29.21 (CH₂, C-6), 32.11 (CH₂, C-4), 60.13 (CH₂, C-11), 121.38 (CH, C-2), 128.67 (CH, C-8), 132.01 (CH, C-9), 149.24 (CH, C-3), 166.75 (C, C-1) ppm.

Scent profile (0.5% in diethyl phthalate): green, complex floral (scent of white flowers), lily-of-the-valley, watery, cyclamen, aldehydic, fatty; afterscent: cucumber, green, watery

Flavour profile (2 ppm in aqueous 5% sucrose or 0.5% sodium chloride solution): juicy, cucumber, green, fatty, soapy, melon, lasting

### Example 3

### Melon Aroma

| Ingredient | Amount in grams |
|---|---|
| Vanillin | 10.0 |
| Ethyl acetate | 150.0 |
| Ethyl butyrate | 200.0 |
| Ethyl propionate | 50.0 |
| 2,6-Nonadienol, 2% in alcohol | 2.0 |
| cis-6-Nonenal, 1% in alcohol | 0.2 |
| Furaneol 15% in 1,2-propylene glycol | 30.0 |
| Methyl dihydrojasmonate | 7.0 |
| beta-lonone | 0.2 |
| Isoamyl acetate | 60.0 |
| Isoamyl alcohol | 8.0 |
| Isobutyl acetate | 100 |
| (2E,8Z)-Undecadienoic acid ethyl ester from | 20 |
| Example 2 | |
| (2E,6Z)-Nonadienal, 1% in 1,2-propylene glycol | 2.0 |
| 6Z-Nonenol | 2.0 |
| 1,2-Propylene glycol | 358.6 |

### Example 4

### Carbonated Soft Drink

| Ingredient | kg | litres |
|---|---|---|
| Sugar syrup, 65% solids content | 153.850 | 116.55 |
| Citric acid solution 50% | 3.000 | 2.460 |
| Melon aroma from Example 3 | 0.200 | 0.250 |
| Carbonated water | 880.740 | 880.740 |
| Total in preparation | 1037.790 | 1000.000 |

The sugar syrup, citric acid solution and melon aroma are mixed together and topped up with carbonated water. The drink obtained in this way is filled into sterilised bottles, sealed and kept in a cool, dark, dry place.

### Example 5

### Perfume Oil Composition

| | Parts by |
|---|---|
| | weight |
| Allyl cyclohexyl propionate | 3 |
| Amyl salicylate | 2 |
| Benzyl acetate | 70 |
| Citral 10% in DPG | 2 |
| Citronellol | 120 |
| Cyclamen aldehyde | 9 |
| Dihydromyrcenol | 3 |
| Dimethylbenzylcarbinyl acetate | 3 |
| Ethyl salicylate 10% in DPG | 2 |
| Eugenol | 3 |
| Indoflor 10% in DPG¹⁾ | 16 |
| Galaxolide 50% in DEP²⁾ | 59 |
| Geraniol | 34 |
| Dihydromethyl jasmonate | 6 |
| Heliotropin | 4 |
| Hexyl cinnamaldehyde | 121 |
| Hydroxycitronellal | 42 |
| Indole | 6 |
| Isobutyl salicylate | 1 |
| Lavandin oil | 6 |
| Lemon oil | 2 |
| Acetyl cedrene | 9 |
| Lilial³⁾ | 190 |
| Linalool | 32 |
| Linalyl acetate | 8 |
| Methyl anthranilate 10% in DPG | 4 |
| Nerol | 8 |
| Orange oil | 50 |
| Phenylacetaldehyde dimethylacetal | 6 |
| Phenyl ethyl alcohol | 54 |
| Sandalwood oil | 3 |
| Tonalide⁴⁾ | 2 |
| Total: | 880 |

¹⁾Trade name of Symrise, Holzminden, Germany
²⁾Trade name of IFF, New Jersey, USA
³⁾Trade name of Givaudin, Zürich, Switzerland
⁴⁾Trade name of PFW, Barneveld, Netherlands

Through the addition of 60 parts by weight of (2E,7Z)-undecadienoic acid ethyl ester from Example 1, the impression of white flowers and the floral character in general are strengthened and rounded out, a slightly green-fruity note being added.

Through the addition of 90 parts by weight of (2E,8Z)-undecadienoic acid ethyl ester from Example 2, the floral impression is emphasised and rounded out, the lily-of-the-valley note in particular being strengthened and green-aldehydic notes being added. In addition, an intensification of the scent (booster effect) is observed.

### Example 6

### Shampoo

The two perfume oil compositions from Example 5 (after addition of (2E,7Z)-undecadienoic acid ethyl ester from Example 1 or (2E,8Z)-undecadienoic acid ethyl ester from Example 2), were incorporated separately in a proportion of 0.5 wt. % each into a basic shampoo matrix with the following composition:

| | |
|---|---|
| Sodium lauryl ether sulfate | 12% |
| (e.g. Texapon NSO, Cognis Deutschland GmbH) | |
| Cocamidopropylbetaine | 2% |
| (e.g. Dehyton K, Cognis Deutschland GmbH) | |
| Sodium chloride | 1.4% |
| Citric acid | 1.3% |
| Phenoxyethanol, methyl, ethyl, butyl | 0.5% |
| and propyl paraben | |
| Water | 82.8% |

The pH of the basic shampoo matrix was around 6. It was used in each case to make 100 ml of a 20 wt. % aqueous shampoo solution. 2 strands of hair were washed together in this shampoo solution for 2 minutes and then rinsed under running hand-hot water for 20 seconds. One hair strand was then packed wet into aluminium foil and the second was dried with a hair dryer. The olfactory quality of all hair strands was assessed by a panel.

TABLE 1
Preferred alkadienoic acid esters having formula (I)

| R1 | R2 | n | Nomenclature name |
|---|---|---|---|
| Methyl | Methyl | 2 | 2,7-Nonadienoic acid methyl ester |
| Methyl | Ethyl | 2 | 2,7-Decadienoic acid methyl ester |
| Methyl | 1-Propyl | 2 | 2,7-Undecadienoic acid methyl ester |
| Methyl | Methyl | 3 | 2,8-Decadienoic acid methyl ester |
| Methyl | Ethyl | 3 | 2,8-Undecadienoic acid methyl ester |
| Methyl | 1-Propyl | 3 | 2,8-Dodecadienoic acid methyl ester |
| Methyl | Methyl | 4 | 2,9-Undecadienoic acid methyl ester |
| Methyl | Ethyl | 4 | 2,9-Dodecadienoic acid methyl ester |
| Methyl | 1-Propyl | 4 | 2,9-Tridecadienoic acid methyl ester |
| Ethyl | Methyl | 2 | 2,7-Nonadienoic acid ethyl ester |
| Ethyl | Ethyl | 2 | 2,7-Decadienoic acid ethyl ester |
| Ethyl | 1-Propyl | 2 | 2,7-Undecadienoic acid ethyl ester |
| Ethyl | Methyl | 3 | 2,8-Decadienoic acid ethyl ester |
| Ethyl | Ethyl | 3 | 2,8-Undecadienoic acid ethyl ester |
| Ethyl | 1-Propyl | 3 | 2,8-Dodecadienoic acid ethyl ester |
| Ethyl | Methyl | 4 | 2,9-Undecadienoic acid ethyl ester |
| Ethyl | Ethyl | 4 | 2,9-Dodecadienoic acid ethyl ester |
| Ethyl | 1-Propyl | 4 | 2,9-Tridecadienoic acid ethyl ester |
| Allyl | Methyl | 2 | 2,7-Nonadienoic acid allyl ester |
| Allyl | Ethyl | 2 | 2,7-Decadienoic acid allyl ester |
| Allyl | 1-Propyl | 2 | 2,7-Undecadienoic acid allyl ester |
| Allyl | Methyl | 3 | 2,8-Decadienoic acid allyl ester |
| Allyl | Ethyl | 3 | 2,8-Undecadienoic acid allyl ester |
| Allyl | 1-Propyl | 3 | 2,8-Dodecadienoic acid allyl ester |
| Allyl | Methyl | 4 | 2,9-Undecadienoic acid allyl ester |
| Allyl | Ethyl | 4 | 2,9-Dodecadienoic acid allyl ester |
| Allyl | 1-Propyl | 4 | 2,9-Tridecadienoic acid allyl ester |
| tert-Butyl | Methyl | 2 | 2,7-Nonadienoic acid tert-butyl ester |
| tert-Butyl | Ethyl | 2 | 2,7-Decadienoic acid tert-butyl ester |
| tert-Butyl | 1-Propyl | 2 | 2,7-Undecadienoic acid tert-butyl ester |
| tert-Butyl | Methyl | 3 | 2,8-Decadienoic acid tert-butyl ester |
| tert-Butyl | Ethyl | 3 | 2,8-Undecadienoic acid tert-butyl ester |
| tert-Butyl | 1-Propyl | 3 | 2,8-Dodecadienoic acid tert-butyl ester |
| tert-Butyl | Methyl | 4 | 2,9-Undecadienoic acid tert-butyl ester |
| tert-Butyl | Ethyl | 4 | 2,9-Dodecadienoic acid tert-butyl ester |
| tert-Butyl | 1-Propyl | 4 | 2,9-Tridecadienoic acid tert-butyl ester |

1. The method of using a composition comprising a fatty acid ester having formula (I), 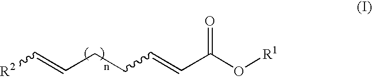
wherein
R¹ is an alkyl radical having 1 to 10 C atoms,
a cycloalkyl radical having 3 to 8 C atoms,
an alkenyl radical having 2 to 10 C atoms or
a cycloalkenyl radical having 3 to 8 C atoms,
R² is an alkyl radical having 1 to 10 C atoms,
n denotes 1, 2, 3, 4 or 5,
and
two double bonds in a fatty acid radical are mutually independently (E)- or (Z)-configured,
as a fragrance and aroma composition.

2. The method according to claim 1 wherein the fatty acid ester having formula (I) comprises preferably between 9 C atoms and 18 C atoms, more preferably between 9 C atoms and 16 C atoms, and most preferably between 10 C atoms and 16 C atoms.

3. The method according to claim 1, wherein for the fatty acid ester having formula (I),
R¹ is an alkyl radical having 1 to 4 C atoms,
a cycloalkyl radical having 3 to 6 C atoms,
an alkenyl radical having 2 to 4 C atoms or
a cycloalkenyl radical having 3 to 6 C atoms,
and
R² is an alkyl radical having 1 to 3 C atoms
and
n denotes 2, 3 or 4.

4. The method according to claim 1, wherein
R² denotes an alkyl radical which is a methyl, ethyl or 1-propyl.

5. The method according to one of claim 1, wherein the fatty acid ester having formula (I) is 2,7-nonadienoic acid methyl ester,
2,7-decadienoic acid methyl ester,
2,7-undecadienoic acid methyl ester,
2,8-decadienoic acid methyl ester,
2,8-undecadienoic acid methyl ester,
2,8-dodecadienoic acid methyl ester,
2,9-undecadienoic acid methyl ester,
2,9-dodecadienoic acid methyl ester,
2,9-tridecadienoic acid methyl ester,
2,7-nonadienoic acid ethyl ester,
2,7-decadienoic acid ethyl ester,
2,7-undecadienoic acid ethyl ester,
2,8-decadienoic acid ethyl ester,
2,8-undecadienoic acid ethyl ester,
2,8-dodecadienoic acid ethyl ester,
2,9-undecadienoic acid ethyl ester,
2,9-dodecadienoic acid ethyl ester,
2,9-tridecadienoic acid ethyl ester,
2,7-nonadienoic acid allyl ester,
2,7-decadienoic acid allyl ester,
2,7-undecadienoic acid allyl ester,
2,8-decadienoic acid allyl ester,
2,8-undecadienoic acid allyl ester,
2,8-dodecadienoic acid allyl ester,
2,9-undecadienoic acid allyl ester,
2,9-dodecadienoic acid allyl ester,
2,9-tridecadienoic acid allyl ester,
2,7-nonadienoic acid tert-butyl ester,
2,7-decadienoic acid tert-butyl ester,
2,7-undecadienoic acid tert-butyl ester,
2,8-decadienoic acid tert-butyl ester,
2,8-undecadienoic acid tert-butyl ester,
2,8-dodecadienoic acid tert-butyl ester,
2,9-undecadienoic acid tert-butyl ester,
2,9-dodecadienoic acid tert-butyl ester, or
2,9-tridecadienoic acid tert-butyl ester.

6. The method according to claim 1, wherein the fatty acid ester having formula (I) is 2,7-decadienoic acid ethyl ester,
2,7-decadienoic acid methyl ester,
2,7-decadienoic acid tert-butyl ester,
2,7-decadienoic acid allyl ester,
2,8-undecadienoic acid ethyl ester,
2,8-undecadienoic acid methyl ester,
2,8-undecadienoic acid tert-butyl ester, or
2,8-undecadienoic acid allyl ester.

7. The method according to claim 1, wherein the fatty acid ester having formula (I) is 2,7-decadienoic acid ethyl ester,
2,7-decadienoic acid methyl ester,
2,8-undecadienoic acid ethyl ester, or
2,8-undecadienoic acid methyl ester.

8. The method according to claim 1, wherein the composition further comprises:
a scent with one or more of the notes green, fruity, watery, floral, aldehydic, fatty, melon, cucumber, apple, violet and lily-of-the-valley for imparting, modifying or strengthening the scent, or
a flavour with one or more of the notes juicy, green, cucumber, peel-like, melon, grape and aldehyde for imparting, modifying or strengthening the flavour.

9. A perfumed or aromatized product comprising:
(a) a fatty acid ester having formula (I), 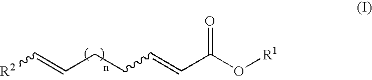
wherein
R¹ is an alkyl radical having 1 to 10 C atoms,
a cycloalkyl radical having 3 to 8 C atoms,
an alkenyl radical having 2 to 10 C atoms or
a cycloalkenyl radical having 3 to 8 C atoms,
R² is an alkyl radical having 1 to 10 C atoms,
n denotes 1, 2, 3, 4 or 5,
and
two double bonds in a fatty acid radical are mutually independently (E)- or (Z)-configured; and
(b) at least one other fragrance or aroma composition,
wherein the perfumed or aromatized product does not include (2E,7E)- or (2E,7Z)-decadienoic acid methyl ester, (2E,7Z)-decadienoic acid ethyl ester, benzene, 5-octenal or methanol.

10. The product according to claim 9, wherein a ratio by weight of a total amount of fatty acid esters having formula (I) to a total amount of other fragrances or aroma compositions is in the range from 1:1000 to 1:0.5.

11. The perfumed or aromatized product according to claim 9, wherein the product contains a total amount of fatty acid esters having formula (I) sufficient to impart, modify or strengthen
(i) a scent note of the type green, fruity, watery, floral, aldehydic, fatty, melon, cucumber, apple, violet and lily-of-the-valley or
(ii) a flavour note of the type juicy, green, cucumber, peel-like, melon, grape and aldehyde.

12. The product according to claim 9, wherein the product is a fragrance or aroma composition.

13. The product according to claim 12, wherein the fragrance composition comprises at least one further fragrance which imparts green, fruity, floral, aldehydic and watery notes; or the aroma composition comprises at least one further aroma substance which imparts green, fruity, juicy and watery notes, more particularly notes of watermelon and cucumber.

14. The perfumed or aromatized product according to claim 13, wherein the product is perfume extracts, eau de parfums, eau de toilettes, aftershaves, eau de colognes, preshave products, splash colognes, perfumed refreshing wipes, acid, alkaline and neutral cleaning agents, textile fresheners, ironing aids, liquid detergents, powdered detergents, laundry pretreatment agents, fabric softeners, laundry soaps, detergent tablets, disinfectants, surface disinfectants, air fresheners, aerosol sprays, waxes and polishes, personal care products, hand creams and lotions, foot creams and lotions, depilatory creams and lotions, aftershave creams and lotions, tanning creams and lotions, haircare products, deodorants and antiperspirants, decorative cosmetic products, candles, lamp oils, incense sticks, insecticides, repellents, or fuels.

15. A fatty acid ester for use as a fragrance and aroma composition, wherein the fatty acid ester is 2Z,7E-decadienoic acid ethyl ester, 2,8-undecadienoic acid ethyl ester or 2,8-undecadienoic acid methyl ester.

16. A process for imparting, modifying and strengthening
(i) a scent note of the type green, fruity, watery, floral, aldehydic, fatty, melon, cucumber, apple, violet and lily-of-the-valley or
(ii) a flavour note of the type juicy, green, cucumber, peel-like, melon, grape and aldehyde,
by adding a sensorially effective amount of a fragrance or aroma composition including a fatty acid ester, wherein the fatty acid ester is 2Z,7E-decadienoic acid ethyl ester, 2,8-undecadienoic acid ethyl ester or 2,8-undecadienoic acid methyl ester.
